(19) United States
(12) Patent Application Publication
(10) Pub. No.: US 2023/0323329 A1
(43) Pub. Date: Oct. 12, 2023

(54) METHOD FOR REMOVING ANTIFOAMING AGENTS FROM A FERMENTATION BROTH

(71) Applicant: BASF SE, Ludwigshafen (DE)

(72) Inventors: Sebastian Schoof, Ludwigshafen (DE); Thomas Kaeding, Lampertheim (DE); Ronja Muenkel, Ludwigshafen (DE)

(21) Appl. No.: 18/023,130

(22) PCT Filed: Sep. 3, 2021
(86) PCT No.: PCT/EP2021/074332

§ 371 Date: Feb. 24, 2023

(30) Foreign Application Priority Data
Sep. 4, 2020 (EP) 20194676.1

(51) Int. Cl. C12N 9/54; C12N 9/14; C12P 21/02

(52) U.S. Cl. CPC C12N 9/54; C12P 21/02; C12Y 304/21062; C12N 9/14

(57) ABSTRACT

The present invention relates to a method for separating a molecule of interest from an antifoaming agent in a fermentation broth comprising said molecule of interest and said antifoaming agent. The method comprises the steps of filtering the fermentation broth at a temperature in the range of 1° C. to 15° C. above the cloud point of the antifoaming agent, and thereby obtaining a first fraction of the fermentation broth comprising the molecule of interest and a second fraction of the fermentation broth comprising the antifoaming agent, wherein the antifoaming agent is a polyalkylene glycol based antifoaming agent. The invention further relates to a process for purifying a molecule of interest from a fermentation broth comprising said method.

The present invention relates to a method for separating a molecule of interest from an antifoaming agent in a fermentation broth broth comprising said molecule of interest and said antifoaming agent. The method comprises the steps of filtering the fermentation broth at a temperature in the range of 1° C. to 15° C. above the cloud point of the antifoaming agent, and obtaining a first fraction of the fermentation broth comprising the molecule of interest and a second fraction of the fermentation broth comprising at least the antifoaming agent, wherein the antifoaming agent is a polyalkylene glycol based antifoaming agent. The invention further relates to a process for purifying a molecule of interest from a fermentation broth comprising said method.

In industrial fermentation unwanted foaming of the fermentation broth during cultivation is often reduced or prevented by the addition of antifoaming agents to the fermentation broth. Although necessary for reducing unwanted foaming, antifoaming agents are known to interfere with downstream processes for purifying the molecules of interest produced in the fermentation process. Therefore, efficient removal of the antifoaming agents is desirable. In particular, antifoaming agents are known to have a negative impact on subsequent filtration steps, such as fouling of filtration membranes, including microfiltration and ultrafiltration membranes, and the reduction of transmembrane fluxes. Moreover, residual antifoaming agents may cause turbidity of final product formulations and may be responsible for material that does not meet the specified requirements of the final product. Overall, non-efficient removal of antifoaming agents can lead to reduced quality of the final product.

U.S. Pat. No. 4,931,397 discloses a method for removing antifoaming agents during processing of microbial fermentation broths that produce heat-labile molecules of interest. The method comprises the addition of a mineral clay to the fermentation broth and subsequent solid/liquid separatory techniques for separating the resulting clay/antifoaming agent complex. However, addition of a mineral clay and removal thereof appears to be labor-intense and involves additional costs. Depending on the molecule of interest, mineral clay addition may be problematic as unwanted complexing of the molecule of interest or parts thereof may occur.

A common method to remove antifoaming agents requires heating the fermentation broth to temperatures around 60° C. in order to remove the antifoaming agents. However, depending on the molecule of interest heating the fermentation broth to temperatures around 60° C. may not be desirable. In particular, with respect to fermentation broths for the production of heat-labile enzymes and/or other heat-labile molecules temperatures around 60° C. may be problematic as the molecule of interest may be damaged and may (partially) lose its function.

WO2010/043520 A1 discloses the removal of antifoaming agents from a yeast cell culture expressing hydrophobin by microfiltration at a temperature above the cloud point of the antifoaming agent. The temperatures applied are about 25° C. above the cloud point of the antifoaming agent, i.e. around 50° C. For the production of heat-labile molecules of interests, such as enzymes, temperatures around 50° C. are usually too high as they may damage the molecule of interest or impart its function.

Therefore, novel methods for removing antifoaming agents from a fermentation broth are desired which overcome the disadvantages and problems of the prior art and which are particularly suitable for fermentation broths producing heat-labile molecules of interest.

The technical problem underlying the present invention may be seen as the provision of means and methods for complying with the aforementioned needs. It can be solved by the embodiments characterized in the claims and herein below.

Therefore, the present invention relates to a method for separating a molecule of interest from an antifoaming agent in a fermentation broth comprising said molecule of interest and said antifoaming agent, the method comprising the following steps:
(a) filtering said fermentation broth at a temperature in the range of 1° C. to 15° C. above the cloud point of the antifoaming agent; and
(b) thereby obtaining a first fraction of the fermentation broth comprising the molecule of interest and a second fraction of the fermentation broth comprising the antifoaming agent, preferably thereby separating the molecule of interest from the antifoaming agent,
wherein the antifoaming agent is a polyalkylene glycol based antifoaming agent.

Thus, preferably, the method for separating a molecule of interest from an antifoaming agent in a fermentation broth comprising said molecule of interest and said antifoaming agent, comprises the steps of
(a) obtaining a first fraction of the fermentation broth comprising the molecule of interest and a second fraction of the fermentation broth comprising the antifoaming agent by filtering said fermentation broth at a temperature in the range of 1° C. to 15° C. above the cloud point of the antifoaming agent; and
(b) thereby separating the molecule of interest from the antifoaming agent,
   wherein the antifoaming agent is a polyalkylene glycol based antifoaming agent.

The antifoaming agent for use in the method according to the present invention is a polyalkylene glycol (PAG) based antifoaming agent. The antifoaming agent suitable for the method of the present invention may have a cloud point in the range of 15° C. to 40° C., preferably in the range of 20° C. to 35° C., more preferably in the range of 25° C. to 30° C.

Suitable (PAG) based antifoaming agents are known in the art, for example in Junker, Biotechnol. Prog., 2007, Vol. 23, 767-784. Preferably, the antifoaming agent is selected from a polypropylene glycol (PPG), such as Polyglycol P-2000 (Dow), Pluriol® P2000 (BASF), a polyethlyene glycol (PEG), such as Pluracol E (BASF), PEG Typ 300 (Carl Roth); a polyalkylene glycol (PAG), such as Struktol® J 647 (Schill+Seilacher), KFO™ 673 (DyStar), UCON™ LB-625 (Dow), KFO™ 402 (DyStar), an ethylene/propylene oxide block copolymer, such as Pluronic® PE6100 (BASF), Pluronic® PE10100 (BASF), Pluronic® PE3100 (BASF), Pluronic® PE8100 (BASF), Pluronic® F68 (BASF), a polyalcohol based on EO/PO block copolymer, such as Struktol® J 650 (Schill+Seilacher), an alkoxylated fatty acid ester, such as Strukol® J 673 (Schill+Seilacher), a polypropylene-polyethylene glycol copolymer (PPG-PEG), mixtures and derivatives thereof, and other non-ionic surfactant antifoams. “Derivatives thereof” include, preferably refer to, for example ethers and/or co-polymers of the afore-mentioned, in particular PAG derivatives, such as Disfoam GD (Nihon Yushi/BASF); PAG-based polyethers such as Breox FMT30 (Intl. Specialty Chemicals/Cognis); PPG-based polyether dispersions such as Antifoam 204 (Sigma); alkyl poly(alkylene glycol) ether blend Bioquest 1110/1120a (Baker Hughes). “Mixtures thereof” include, preferably refer to, for example blends of the aforementioned for example PAG-based blends such as Antifoam 289, 286 (Sigma), VP1133 (Wacker-Chemie); XFO-371 (Ivanhoe); Sag 5693, 5698 (Union Carbide, OSI specialities). More preferably, the antifoaming agent is a PPG, a PEG, a PAG, an ethylene/propylene oxide block copolymer, a polyalcohol based on EO/PO block copolymer, an alkoxylated fatty acid ester, a PPG-PEG, or a mixture and/or derivative of any of the aforesaid. Still more preferably, the antifoaming agent is a PPG, a PEG, a PAG, an ethylene/propylene oxide block copolymer, a polyalcohol based on EO/PO block copolymer, an alkoxylated fatty acid ester, or a PPG-PEG. Most preferably, the antifoaming agent is a PPG, a PEG, a PAG, or a PPG-PEG.

Preferably, the antifoaming agent has an average molar mass in the range of 500 to 5000 g/mol, more preferably in the range of 1000 to 3000 g/mol, still more preferably about 2000 g/mol, most preferably 2000 g/mol. A particularly preferred antifoaming agent is a PPG having an average molar mass in the range of 500 to 5000 g/mol, more preferably in the range of 1000 to 3000 g/mol, still more preferably about 2000 g/mol, most preferably 2000 g/mol, for example Pluriol® P2000 (BASF) having an average molar mass of approximately 2000 g/mol.

Alternatively, antifoaming agents comprising a silicone based oil and/or a silicone based emulsion may be used. Typically, these comprise poly(dimethylsiloxane) in varying amounts, e.g. from 10% to 100% (v/v). Typical examples thereof may include Sag M-10 (Dow Corning); Sag 471 (Union Carbide); DC-A, A (Dow Corning); Antifoam A (concentrate) (Sigma); Mazu DF100, DF1005 (Noveon, Mazur); FD 101 (Stepan); Q7-2243 LVA (Dow Corning); C100F (Basildon); S184 (Wacker Chemie); Q10-335 (Dow Corning); TMA812 (Toshiba); Antaphron NM40 (UK); Biospumex FDA 165K (Cognis); KM-70, M-7W-0 (Shinetsu Kogaku); Mazu DF 210, 210 S, 2105 (Mazur/Basf/Noveon); 1510 (Dow Corning); 1520 (Dow Corning); AF (Dow Corning); C (Dow Corning) DC-B (Dow Corning); FG-10 (Dow Corning); DSP (Dow Corning); RD (Dow Corning); M30 (Dow Corning); Q7-2587 (Dow Corning); Hodag FD-62 (Lambent); Hodag FD-82 K (Lambent); A (A-5758) (Sigma); B (Sigma, JT Baker); C (Sigma); Chemax DF-(Rutgers); Chemax DF-30 (Rutgers); SE15 (Sigma); SE9 (Wacker Chemie); Sag 10 (Union Carbide); Sag 30 (Union Carbide); Silcolapse 5001 (ACC/Rhodia); Silcolapse 5000 (ICI/Ambersil/Rhodia); GE 60 (GE); Roth 0865 (Carlroth.de); PD30 (Basildon); RS-70426 (Rhodia); 1705-W (Lion); Assaff III (Rhone-Poulenc).

In addition, the present invention relates to a process for purifying a molecule of interest from a fermentation broth comprising the method for separating a molecule of interest from an antifoaming agent in a fermentation broth comprising said molecule of interest and said antifoaming agent disclosed herein.

Unless otherwise noted, the terms used herein are to be understood according to conventional usage by those of ordinary skill in the relevant art.

As used in this specification and in the appended claims, the singular forms of “a” and “an” also include the respective plurals unless the context clearly dictates otherwise. Hence, “a” or “an” can mean one or more, depending upon the context in which it is used. Thus, for example, reference to “a cell” can mean that at least one cell can be utilized.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The inventors surprisingly found that separating a molecule of interest from an antifoaming agent in a fermentation broth comprising said molecule of interest and said antifoaming agent can be achieved by filtration at a temperature in the range of 1° C. to 15° C. above above the cloud point of the antifoaming agent, preferably a range of 1° C. to 15° C. above above the cloud point of the antifoaming agent refers to a temperature within a range of 20° C. to 45° C.

In particular, the method according to the present invention relates to a down-stream process that may be utilized in a process for purifying the molecule of interest from the fermentation broth. “Down-stream process” may be understood as a method or method steps that are performed after the cultivation of the fermentation broth, for example when a predefined or desirable titer of the molecule of interest has been reached.

The terms “recovering” or “purifying” may be used interchangeably and are intended to mean “rendering more pure”. They refer to a process in which the molecule of interest is separated from other compounds or cells present in the fermentation broth.

The term “titer of a molecule of interest” as used herein is understood as the amount of molecule of interest in g per volume of fermentation broth in liter or in g per kg fermentation broth. The titer of a molecule of interest may reach 2 to 100 g molecule/kg fermentation broth at the end of the fermentation process, e.g. at the time of harvest. Preferably, at the time of harvest the titer of a molecule of interest reaches up to 60 g product/kg fermentation broth. More preferably, at the time of harvest the titer of a molecule of interest reaches 5 to 30 g product/kg fermentation broth, more preferably the molecule of interest reaches 8 to 20 g product/kg fermentation broth at the time of harvest. The term “time of harvest” may particularly refer to the end of the fermentation, more particularly to the point in time when a predefined or desirable titer of the molecule of interest has been reached, such as 2 to 100 g molecule of interest/kg fermentation broth. Even more particularly the time of harvest refers to the point in time prior to step (a) of the method according to the invention.

Without wishing to be bound by theory, selecting the temperature of the fermentation broth to temperatures above the cloud point of the antifoaming agent is thought to exploit the phase transition of the antifoaming agent leading to its aggregation. The antifoaming agent may then be separated by filtration or even other separatory means such as sedimentation or centrifugation, preferably by filtration as explained in further detail elsewhere herein.

It is thought that already during the fermentation process, that may preferably take place at temperatures above the cloud point (CP) of the antifoaming agent, a phase separation of the antifoaming agent may occur. However, as the fermentation broth usually is a suspension, the phase separation may remain without consequences. During downstream processing a part of the antifoaming agent may adhere onto the biomass and may be separated therewith, however another part of the antifoaming agent may be solubilized and could hence be unintentionally transferred to further down-stream processing steps. Adequate removal is required in order to avoid fouling of microfiltration or ultrafiltration membranes or other unwanted effects of high levels of antifoaming agents during downstream processing.

Therefore, the invention relates to a method for separating a molecule of interest from an antifoaming agent in a fermentation broth comprising said molecule of interest and said antifoaming agent, the method comprising the following steps:
(a) filtering said fermentation broth at a temperature in the range of 1° C. to 15° C. above the cloud point of the antifoaming agent; and
(b) thereby obtaining a first fraction of the fermentation broth comprising the molecule of interest and a second fraction of the fermentation broth comprising the antifoaming agent, preferably thereby separating the molecule of interest from the antifoaming agent,
   wherein the antifoaming agent is a polyalkylene glycol based antifoaming agent.

The term “antifoaming agent” refers to a chemical molecule capable of inhibiting the formation of foam or reducing the amount of foam during the fermentation process, in particular when cultivating the cells. A typical antifoaming agent according to the present invention is a polyalkylene glycol based antifoaming agent, preferably selected from a polypropylene glycol (PPG), a polyethlyene glycol (PEG), an ethylene/propylene oxide block copolymer, a polyalcohol based on EO/PO block copolymer, an alkoxylated fatty acid ester, a polypropylene-polyethylene glycol copolymer (PPG-PEG), mixtures and derivatives thereof, more preferably a PPG, PEG, or PPG-PEG; even more preferably a PPG, PEG, or PPG-PEG having an average molar mass in the range of 500 to 5000 g/mol. An still even more preferred antifoaming agent according to the present invention is PPG, still even more preferred PPG with an average molar mass of around 2000 g/mol, such as Pluriol® P2000 commercially available from BASF.

Alternatively, antifoaming agents comprising a silicone based oil and/or a silicone based emulsion may be used, as specified elsewhere herein.

The term “cloud point” with respect to the present invention relates to a temperature or a temperature range at which the antifoaming agent, in particular a solution or mixture thereof, starts to phase-separate and two phases may become visible. Hence, at the temperature or the temperature range of the cloud point, the antifoaming agent, in particular an aqueous solution or mixture thereof, may start to become cloudy. This may be referred to as the onset of turbidity. It is thought that at the cloud point and temperatures above, the molecules of the antifoaming agent form aggregates that scatter light which lead to turbidity. This behavior is particularly characteristic of non-ionic surfactants containing polyalkylene chains, which exhibit reverse solubility versus temperature behavior in water and therefore “cloud out” at some point as the temperature is raised. More particularly, the “cloud point” may refer to the temperature at which a solution of the antifoaming agent shows turbidity. Typically, the onset of turbidity occurs when raising the temperature of the solution to temperatures above the cloud point of the antifoaming agent. This turbidity is a reversible process, so that the solution clears again on cooling. The cloud points of antifoaming agents suitable for use in the present invention preferably refer to temperatures in the range of 15° C. to 40° C., preferably in the range of 20° C. to 35° C., more preferably the antifoaming agent may have a cloud point between 25° C. and 30° C. Even more preferably, the cloud point of the antifoaming agent may be understood as referring to a temperature range, e.g. not to a specific temperature, for example to a range within 20° C. to 28° C. The temperature range of the cloud point may depend on the concentration of the antifoaming agent present in the solution, for example at a concentration of 0.1 g/I, the cloud point may lie between 27° C. and 28° C., and/or at a concentration of 1.0 g/I, the cloud point may lie between 22° C. and 23° and/or at a concentration of 2.5 g/I, the cloud point may lie between 20° C. and 21°. Hence, suitable antifoaming agents according to the present invention may refer to polyalkylene glycol based antifoaming agents and may have a cloud point as specified above. Methods for determining the cloud point of an antifoaming agent are known in the art and respective values are usually provided by the manufacturer or distributor. Preferably, the cloud point is determined as specified herein in the Examples; preferably, the cloud point is determined at the concentration of the antifoaming agent used in the fermentation broth, more preferably at a concentration of 1 g/L; most preferably the cloud point of the antifoaming agent is determined at a concentration of the antifoaming agent of 1 g/L in deionized water; also preferably, the temperature or temperature range at which an increase of the measured turbidity of at least 100%, preferably at least 200%, preferably at least 300% is observed, is the cloud point, preferably the increase of turbidity is measured in solution of the antifoaming agent in deionized water.

Commonly, the fermentation broth may contain the antifoaming agent in an amount of 0.1 to 50 g/kg based on the total weight of the fermentation broth, preferably 0.50 to 10 g/kg, more preferably 0.50 to 3 g/kg, based on the total weight of the fermentation broth. The antifoaming agent may be dosed into the fermentation broth prior to and/or during the fermentation process.

The term “fermentation broth” or “culture broth” is used in a broad sense to include any and all fermentation medium during or after fermentation, i.e. during or after contacting with recombinant and/or non-recombinant cells, including downstream products thereof comprising at least a molecule of interest and an antifoaming agent, as well as aliquots and fractions thereof. Preferably, the fermentation broth is the fermentation medium comprising recombinant and/or non-recombinant cells, which are cultivated to express or produce the molecule of interest. In particular, the recombinant or non-recombinant cells may secrete the molecule of interest into the fermentation medium. Hence, the fermentation broth comprising the molecule of interest may refer to a fermentation broth comprising cells that produce the molecule of interest, in particular producing the molecule of interest during cultivation of the cells or during the fermentation process. Thus, in the methods and processes specified herein, the complete volume of fermentation broth obtained in a fermentation may be used, but also only a part thereof. Preferably, the fermentation broth is the liquid as it is obtained at the end of a fermentation process without any further treatments. However, as used herein, the aforesaid terms also include the fermentation broth as obtained in the fermentation process, as well as a product obtained therefrom by removal of cells and/or cell fragments; the latter may also be referred to as “cell-free fermentation broth”. In accordance, the terms “fermentation broth” or “culture broth” also include aliquots. i.e. sub-portions, of the fermentation broth, as well as fractions of the fermentation broth, i.e. parts of the fermentation broth obtained by removal of parts of its constituents; thus the fermentation broth may also be a cell-free fraction of the fermentation broth initially obtained. Preferably, the fermentation broth is a cell-containing fermentation broth, a cell-free fermentation broth, or and aliquot and/or fraction thereof. Preferably, the fermentation broth according to the present invention is free of any mineral clay and/or further compound for complexing, binding or removing the antifoaming agent. More preferably, no mineral clay and/or further compound for complexing, binding or removing the antifoaming agent is added to the fermentation broth in the method according to the present invention.

Thus, the term “fraction of the fermentation broth” or “fraction of the culture broth” may denote only part of the fermentation broth obtained by removal of part of its constituents. Said fraction(s) of the fermentation broth may be obtained by filtration, preferably by microfiltration, or by centrifugation, preferably by decanter centrifugation, disc stack centrifugation or a nozzle separator. Fractions of the fermentation broth may be obtained by centrifugation of the fermentation broth leading to a supernatant, a fraction comprising the liquid part, and a spin-down fraction. The term “a spin down fraction of a fermentation broth” or “a spin down fraction of a culture broth” denotes the fraction of a fermentation broth obtained after centrifugation, including cells, insoluble substrates, and insoluble fermentation products. Alternatively, the fractions of the fermentation broth may be obtained by filtration of the fermentation broth leading to a filtrate or permeate comprising the liquid fraction and a retentate comprising the biomass comprising the cells and parts thereof and/or other insoluble (solid fraction). Fractions may also include parts of the fermentation broth, if not the complete fermentation broth present in the fermenter is harvested for the recovery of the molecule of interest. In particular, in the method of the invention, for example after filtering in step (a), the fraction of the fermentation broth comprising the molecule of interest may be a liquid fraction, a filtrate, and the second fraction, a retentate, comprising at least the antifoaming agent and/or the biomass or parts of the biomass may be a solid fraction. According to the method of the present invention, the antifoaming agent may preferably be separated from the fraction of the fermentation broth comprising the molecule of interest by filtration. Preferably, the fraction(s) of the fermentation broth may be selected from (i) the spin-down fraction (solid fraction) obtained by centrifugation of the fermentation broth, and (ii) the supernatant (liquid fraction) obtained by centrifugation of the fermentation broth.

More preferably, the fractions of the fermentation broth may be selected from (i) the liquid pass-through fraction, also referred to as filtrate, and (ii) the retained fraction, also referred to as retentate, obtained by filtration of the fermentation broth. Preferably, the retentate is a solid fraction; it may, however, depending on filtration conditions, also be liquid.

A “fermentation process” comprises the cultivation of cells in a suitable fermentation medium, also referred to as “cultivation medium”. “Cultivation of the cells” or “growth of the cells” is not understood to be limited to an exponential growth phase, but can also include the physiological state of the cells at the beginning of growth after inoculation and during a stationary phase. Typically the cultivation may take place at a cultivation temperature suitable for supporting growth of the cells. More typically, the cultivation temperature may lay within the range of 25° C. to 45° C. Further details are specified elsewhere herein. The fermentation process may usually take place in a fermenter, preferably in a fermenter with a volume scale which is at least 1 m3.

The present invention, provides for a method that can be applied for culturing microbial cells in both, laboratory and industrial scale fermentation processes. “Industrial fermentation” as referred to in accordance with the present invention preferably refers to a cultivation method in which at least 200 g of a carbon source per liter of initial fermentation medium will be added. Preferably, an industrially relevant fermentation process encompasses a fermentation process on a volume scale which is 1-500 m3 with regard to the nominal fermenter size, preferably 5-500 m3, more preferably 10-500 m3, even more preferably 25-500 m3, most preferably 50-500 m3. In other words, an industrially relevant fermentation process encompasses a fermentation process on a volume scale which is preferably at least 1000 L with regard to the nominal fermenter size, preferably at least 5,000 L, more preferably at least 10,000 L, even more preferably at least 25,000 L, still even more preferably at least 50,000 L and most preferably 50,000-500,000 L.

The term “fermentation medium” also referred to as “cultivation medium” relates to a water-based solution containing one or more chemical compounds that can support the growth of cells. Preferably, the fermentation medium according to the present invention is a complex fermentation medium or a chemically defined fermentation medium. Any medium suitable for the culture of the particular cell may be used as fermentation medium. The fermentation medium may be a minimal medium as described before, e.g., in WO 98/37179, or the fermentation medium may be a complex medium comprising complex nitrogen and/or carbon sources, wherein the complex nitrogen source may be partially hydrolyzed as described in WO 2004/003216. Furthermore, the fermentation medium may contain a phosphate and/or carbonate source. According to the preset invention, the fermentation medium may preferably comprise at least one antifoaming agent.

The term “separating a molecule of interest from an antifoaming agent in a fermentation broth” may be understood to refer to separating the antifoaming agent at least partially from the fraction of the fermentation broth comprising the molecule of interest. Preferably, the term relates to obtaining at least one fraction of the fermentation broth comprising the molecule of interest in which the molar ratio and/or the mass ratio of molecule of interest to antifoaming agent is increased compared to the state before separation. Thus, the term may relate to decreasing the relative concentration of antifoaming agent in the fraction of the fermentation broth comprising the molecule of interest, to increasing the relative concentration of the molecule of interest in the fraction of the fermentation broth comprising the molecule of interest, or to both decreasing the relative concentration of antifoaming agent and increasing the relative concentration of the molecule of interest in the fraction of the fermentation broth comprising the molecule of interest. In particular, the term may refer to obtaining a fraction of the fermentation broth comprising the molecule of interest and a second fraction comprising at least the antifoaming agent and preferably the biomass, or a part of the biomass. More particularly, separating a molecule of interest from an antifoaming agent in a fermentation broth may result in obtaining a filtrate, also referred to as permeate, containing the molecule of interest and a retentate, or filter cake, containing the antifoaming agent and/or the biomass, for example when microfiltration at a temperature above the cloud point is applied. Alternatively, separating a molecule of interest from an antifoaming agent in a fermentation broth may result in obtaining a filtrate, also referred to as permeate, containing the antifoaming agent and a retentate containing the molecule of interest, depending on the type of filtration applied, for example in the case of ultrafiltration at a temperature below the cloud point. As the skilled person understands, the fraction comprising the antifoaming agent may comprise some molecule of interest as well; thus, preferably, the fraction comprising the molecule of interest is the fraction comprising the higher molar ratio and/or mass ratio of molecule of interest to antifoaming agent available at the respective step; in case more than two fractions are obtained, the fraction(s) comprising the molecule of interest preferably is/are the at least one fraction(s) comprising the highest molar ratio and/or mass ratio of molecule of interest to antifoaming agent available at the respective step. Thus, preferably, the fraction(s) comprising the antifoaming agent preferably is/are the fraction(s) comprising the lowest molar ratio and/or mass ratio of molecule of interest to antifoaming agent available at the respective step.

The second fraction comprising at least the antifoaming agent may be understood as comprising at least 50% of the total amount of antifoaming agent initially present in the fermentation broth prior to the filtering step (a), preferably at least 60% more preferably at least 70%, even more preferably at least 80%, at least 85% or at least 90%. The second fraction may in addition comprise the biomass or parts thereof, in particular the biomass or parts thereof may refer to at least 50% of the total amount of biomass initially present in the fermentation broth prior to the filtering step (a), preferably at least 60% more preferably at least 70%, even more preferably at least 80%, at least 85% or at least 90%. The term “initially present” may in particular refer to the amount of the biomass and/or the antifoaming agent at the time of harvest.

The term “biomass” refers to recombinant or non-recombinant cells which produce the desired molecules of interest and fragments or parts of these cells present in the fermentation broth.

According to the invention, the fermentation broth may comprise recombinant and/or non-recombinant cells producing the molecule of interest; preferably the cells are microbial cells. Suitable microbial cells according to the invention may include for example archeae, fungi including yeasts, and bacteria. Particularly suitable according to the present invention are bacterial cells.

Still alternatively, the cells producing the molecule of interest may also be a eukaryote, such as a mammalian cell, an insect cell, or a plant cell.

For the purposes of the invention, “recombinant” (or transgenic) with regard to a cell means that the cell contains a polynucleotide which is introduced by man by gene technology and with regard to a polynucleotide the term “recombinant” includes all those constructions brought about by man by gene technology/recombinant DNA techniques in which either
(a) the sequence of the polynucleotide or a part thereof, or
(b) one or more genetic control sequences which are operably linked with the polynucleotide, including, but not limited thereto, a promoter, or
(c) both a) and b) are not located in their wildtype genetic environment or have been modified.

The term “non-recombinant” (or native or wildtype or endogenous) cell and “non-recombinant” (or native or wildtype or endogenous) polynucleotide or polypeptide refers to the cell as found in nature and to the polynucleotide or polypeptide in question as found in a cell in its natural form and genetic environment, respectively (i.e., without there being any human intervention).

Bacterial cells may include gram-positive or gram-negative bacteria. The bacterial cells may be selected from the group consisting of Escherichia, Buttiauxella and Pseudomonas, Bacillus, Brevibacterium, Corynebacterium, Streptococcus, Streptomyces, Staphylococcus, Enterococcus, Lactobacillus, Lactococcus, Clostridum, Geobacillus, and Oceanobacillus cells. According to the present invention Bacillus cells may be preferred; even more preferred are Bacillus cells selected from the group including, but not limited to, Bacillus alkalophilus, Bacillus amyloliquefaciens, Bacillus brevis, Bacillus circulars, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells. Still even more preferably, the cell is a Bacillus amyloliquefaciens, Bacillus lentus, Bacillus licheniformis, Bacillus stearothermophilus or Bacillus subtilis cell. Alternatively, the bacterial cell is a Bacillus licheniformis cell or a Bacillus subtilis cell. Still even more preferably, the Bacillus cell is a Bacillus cell of Bacillus subtilis, Bacillus pumilus, Bacillus licheniformis, or Bacillus lentus. Most preferably, the cell is a Bacillus licheniformis cell. Even more preferably, the Bacillus licheniformis cell is selected from the group consisting of Bacillus licheniformis cells as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.

Typically, the host cell belongs to the species Bacillus licheniformis, such as a host cell of the Bacillus licheniformis strain as deposited under American Type Culture Collection number ATCC 14580 (which is the same as DSM 13, see Veith et al. “The complete genome sequence of DSM 13, an organism with great industrial potential.” J. Mol. Microbiol. Biotechnol. (2004) 7:204-211). Alternatively, the host cell may be a host cell of Bacillus Bacillus licheniformis licheniformis strain ATCC 53926. Alternatively, the host cell may be a host cell of Bacillus licheniformis strain ATCC 31972. Alternatively, the host cell may be a host cell of Bacillus licheniformis strain ATCC 53757. Alternatively, the host cell may be a host cell of Bacillus licheniformis strain ATCC 53926. Alternatively, the host cell may be a host cell of Bacillus licheniformis strain ATCC 55768. Alternatively, the host cell may be a host cell of Bacillus licheniformis strain DSM 394. Alternatively, the host cell may be a host cell of Bacillus licheniformis strain DSM 641. Alternatively, the host cell may be a host cell of Bacillus licheniformis strain DSM 1913. Alternatively, the host cell may be a host cell of Bacillus licheniformis strain DSM 11259. Alternatively, the host cell may be a host cell of Bacillus licheniformis strain DSM 26543.

Preferably, the Bacillus licheniformis strain is selected from the group consisting of Bacillus licheniformis ATCC 14580, ATCC 31972, ATCC 53757, ATCC 53926, ATCC 55768, DSM 13, DSM 394, DSM 641, DSM 1913, DSM 11259, and DSM 26543.

Alternatively, the microbial cell may be a fungal cell selected from the phyla Ascomycota, Basidiomycota, Chytridiomycota, Zygomycota, and Oomycota, as well as all mitosporic fungi and Saccharomycoideae. Preferably, the fungal cell is selected from the group consisting of Aspergillus, Penicillium, Candida, Trichoderma, Thermothelomyces, in particular Thermothelomyces thermophila (also referred to as Myceliophthora thermophila), and Pichia.

Preferably, the molecule of interest is filterable, preferably by microfiltration. Thus, the molecule of interest preferably has an average outer diameter of less than 2 μm, preferably less than 0.2 μm, more preferably less than 0.1 μm. Thus, the molecule of interest preferably has an average molecular mass of less than 5000 kDa, preferably less than 1000 kDa, more preferably less than 500 kDa. As will be understood, the molecule of interest may also be a complex of non-covalently connected molecules, e.g. a homo- or heteromultimeric polypeptide.

Preferably, the molecule of interest is heat-labile. More preferably, the molecule of interest is selected from the group consisting of proteins, amino acids, fatty acids, vitamins, coenzymes, polyhydroxyalcanoates, organic acids, antibiotics, alcohols, terpenes, nucleotides, steroids, carotenoids, and polysaccharides, small molecules. More preferably, the molecule of interest is a protein, even more preferably an enzyme, still even more preferably a heat-labile enzyme. The molecule of interest may be referred to as the fermentation product, e.g. the product produced by the recombinant or non-recombinant cells in the fermentation broth.

The term “heat-labile molecule of interest” refers to molecules of interest that are instable or that are impaired in function when exposed to temperatures above 45° C., more preferably to temperatures above 50° C., even more preferably to temperatures above 60° C., or above 70° C.

Preferably, the molecule of interest is an enzyme. The enzyme may be selected from the group consisting of hydrolases (EC 3), preferably is a glycosidase (EC 3.2), an esterase (EC 3.1) peptidases, or a and protease (EC 3.4). More preferably the enzyme may be selected from amylase, in particular alpha-amylase (EC 3.2.1.1), glucoamylase, pullulanase, metalloprotease, lipase (EC 3.1.1.3), cutinase, acyl transferase, cellulase (EC 3.2.1.4), endoglucanase, cellubiohydrolase, xylanase, xyloglucantransferase, xylosidase, mannanase (EC 3.2.1.25), phytase (EC 3.1.3.8), a nuclease (EC 3.1.11 to EC 3.1.31), phosphatase, xylose isomerase, glucoase isomerase, lactase (EC 3.2.1.108), acetolactate decarboxylase, pectinase, pectin methylesterase, polygalacturonidase, lyase, pectate lyase, arabinase, arabinofuranosidase, galactanase, a laccase, peroxidase and an asparaginase. Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lactase (EC 3.2.1.108), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, glucoamylase, nuclease, and cellulase, preferably, amylase or protease, preferably, a protease. More preferred, the enzyme is selected from protease, amylase, lactase, and mannanase, in particular preferred, selected from protease, amylase, and lactase. Most preferred is a protease, preferably, a serine protease (EC 3.4.21), preferably a subtilisin protease.

The molecule of interest may be secreted into the fermentation broth, or may be brought into solution by other means. Preferably, the molecule of interest is secreted by the recombinant or non-recombinant cells into the fermentation broth. Secretion of the molecule of interest into the fermentation medium may allow for separation of the molecule of interest from the fermentation broth. In particular, in the case where the molecule of interest is a protein, the nucleic acid construct used for expressing the protein of interest in the recombinant or non-recombinant cells comprises a polynucleotide encoding for a signal peptide that directs secretion of the protein of interest into the fermentation medium. Various signal peptides are known in the art. Preferred signal peptides are selected from the group consisting of the signal peptide of the AprE protein from Bacillus subtilis or the signal peptide from the YvcE protein from Bacillus subtilis.

The method according to the invention may comprise cultivating the fermentation broth, in particular cultivating the recombinant or non-recombinant cells in the fermentation broth producing the molecule of interest, in the presence of the antifoaming agent. Advantageously, the formation of foam during cultivation is inhibited or reduced due to the presence of the antifoaming agent. Preferably, cultivation takes place at a cultivation temperature above the cloud point of the antifoaming agents. More preferably, a cultivation above the cloud point of the antifoaming agent refers to a cultivation temperature in the range of 25° C. and 45° C., even more preferably in the range of 27° C. to 40° C., still even more preferably of 27° C. to 37° C.

Preferably, the cultivation of the fermentation broth, in particular cultivation of the cells in the fermentation broth producing the molecule of interest may be performed prior to step (a) and/or (b).

The pH of the fermentation broth may be adjusted depending on the microbial cell. Commonly, the pH may be adjusted to a pH of between 6.6 and 9, preferably to 6. As an example, if a Bacillus cell is used, the pH is adjusted to or above pH 6.8, pH 7.0, pH 7.2, pH 7.4, or pH 7.6. Preferably, the pH of the fermentation broth during cultivation of the Bacillus cells is adjusted to pH 6.8 to 9, preferably to pH 6.8 to 8.5, more preferably to pH 7.0 to 8.5, most preferably to pH 7.2 to pH 8.0.

Step (a) of the method according to the invention comprises filtering the fermentation broth or a fraction thereof comprising at least the antifoaming agent and a molecule of interest at a temperature in the range of 1° C. to 15° C. above the cloud point of the antifoaming agent. Preferably, a fraction of the fermentation broth with respect to step (a) denotes only part of the fermentation broth when only a part of the fermentation broth is harvested. More preferably, the fermentation broth or a fraction thereof subjected to step (a) still comprises the cells producing the molecule of interest, also referred to as the biomass.

By the filtration in step (a) the antifoaming agent may be separated with the solid fraction while the molecule of interest may be obtained in the liquid fraction. Preferably, the second fraction, i.e. the retentate fraction comprises the antifoaming agent and the biomass. This is advantageous as only a single filtration step is required in order to remove both, the biomass and the antifoaming agent from the fermentation broth and to achieve efficient separation of the antifoaming agent from the fermentation broth from the molecule of interest.

A temperature above the cloud point may be understood as a temperature that is at least 1° C. above the cloud point of the antifoaming agent, preferably at least 2° C., more preferably at least 5°, even more preferably at least 10° C. above the cloud point of the antifoaming agent. The filtering in step (a) is performed at a temperature in the range of 1° C. to 15° C. above the cloud point of the antifoaming agent, preferably at a temperature in the range of 5° C. to 15° C. above the cloud point of the antifoaming agent, more preferably at a temperature in the range of 7° C. to 12° C. above the cloud point of the antifoaming agent, even more preferably in the range of 10° C. to 12° C. above the cloud point of the antifoaming agent. Particularly, a temperature in the range of 1° C. to 15° C. above the cloud point of the antifoaming agent refers to a temperature in the range of 20° C. to 45° C., more particularly it refers to a temperature in the range of 25° C. to 40° C., even more particularly to a temperature in the range of 28° C. to 35° C. Temperatures above 45° C. in the filtering step (a) are not desirable as they may damage the molecule of interest or its function.

Advantageously, it has been found that filtering at a temperature in the range of 1° C. to 15° C. above the cloud point of the antifoaming agent results in efficient removal of the antifoaming agent and hence efficient separation of the antifoaming agent from the molecule of interest. The temperature during filtering may usually be at most 15° C. above the cloud point of the antifoaming agent, particularly the temperature should not exceed 45° C., otherwise the temperature may cause harm to the molecule of interest, in particular to a heat-labile molecule of interest. At temperatures lower than specified above, the removal of the antifoaming agent may not be efficient as the phase separation of the antifoaming agent may not be sufficient in order to support efficient retention of the antifoaming agent in the filtering step (a) and hence to support efficient separation of the antifoaming agent from the molecule of interest. Advantageously, the choice of the temperature in step (b) may be adapted within the above-specified range depending on the cloud point of the antifoaming agent present in the fermentation broth and the temperature sensitivity of the molecule of interest.

Preferably step (a) may be performed at a temperature within a range of 20° C. to 45° C.; more preferably at a temperature between 25° C. and 40° C., even more preferably at a temperature between 28° C. and 37° C., still even more preferably filtering takes place in the range of 30° C. to 35° C.

Adjusting the temperature may be performed by any techniques known to the person skilled in the art; in particular, adjusting the temperature may involve cooling or heating the fermenter, the tank, the feed and/or the filtering device containing the fermentation broth or the fraction thereof using standard procedures and equipment.

Preferably, the temperature is maintained throughout the filtering in the desired range. The temperature of the fermentation broth may be set to the desired range prior to the filtering step by adjusting the temperature of the respective tank or container comprising the fermentation broth or the fraction thereof designated for filtering. The time required for filtering may be kept sufficiently short, such that the temperature of the fermentation broth or the fraction thereof can be maintained throughout the filtering process. Maintaining the temperature in step (a) within the desired range throughout the filtering advantageously may lead to efficient separation of the antifoaming agent from the molecule of interest.

In addition, the separation equipment such as for example the filter, and/or piping guiding the fraction of the fermentation broth into the separation equipment may be heated or cooled to achieve and/or maintain the desired temperature in the filtration step (a).

The antifoaming agent may be dosed into the fermentation broth prior to step (a), preferably the antifoaming agent may be dosed into the fermentation broth during the fermentation process and/or at the beginning of the fermentation process. The amount of antifoaming agent in the fermentation broth may amount up to 0.1 to 50 g/kg based on the total weight of the fermentation broth. Preferably, the amount of antifoaming agent in the fermentation broth may amount up to 0.5 to 10 g/kg based on the total weight of the fermentation broth, more preferably 0.50 to 3 g/kg, based on the total weight of the fermentation broth.

Simultaneously with or prior to step (a) the method may further comprise the addition of filtering aids, the adjusting of the pH and/or a heating step. Preferably, the method according to the invention does not comprise the addition of any flocculating agent.

Further, in order to ensure thorough mixing and/or uniform temperature adjustment, the fermentation broth may be constantly stirred over a predetermined incubation time, in particular the fermentation broth may be stirred prior to step (a) and/or (c) of the method according to the invention. The incubation time may vary from 1 to 120 minutes. The incubation time may be 1 minute, 10 minutes, 20 minutes, 30 minutes, 40 minutes, 50 minutes, 60 minutes, 70 minutes, 80 minutes, 90 minutes, 100 minutes, 110 minutes, 110 minutes, or 120 minutes. Preferably, the incubation time is between 60-90 minutes, most preferably, the incubation time is 60 minutes. Alternatively, the pH is adjusted by in-line addition of the base.

The filtering in step (a) preferably comprises microfiltration. Microfiltration may be performed using techniques and equipment known to the person skilled in the art. In particular, the filtering in step (a) may comprise dead-end filtration or cross flow filtration (tangential flow filtration), preferably involving microfiltration membranes or microfiltration membrane modules. Thus, the filtering step preferably comprises cross-flow microfiltration or dead-end microfiltration, more preferably comprises cross-flow microfiltration. Suitable microfiltration membranes or membrane modules may comprise filter membranes with pore sizes in the range of 0.05 to 10 μm, preferably 0.05 to 2 μm, more preferably 0.05 to 0.3 μm, depending on the cells producing the molecule of interest. Preferably, the cell producing the molecule of interest is a eukaryotic cell, preferably as specified elsewhere herein, and the pore size is in the range of from 0.05 μm to 25 μm, more preferably 0.1 μm to 20 μm, still more preferably 0.5 μm to 15 μm, most preferably 1 μm to 10 μm. More preferably, the cell producing the molecule of interest is a prokaryotic cell, preferably as specified elsewhere herein, and the pore size is in the range of from 0.02 μm to 5 μm, more preferably 0.05 μm to 2 μm, still more preferably 0.1 μm to 1 μm, even more preferably 0.15 μm to 0.9 μm, most preferably 0.2 μm. However, other pore sizes may be envisaged as well, e.g. in dead end filtration, in which filtration parameters may eventually be determined by the filtration cake.

Step (b) of the method comprises obtaining a first fraction of the fermentation broth comprising the molecule of interest and a second fraction of the fermentation broth comprising the antifoaming agent and preferably the biomass.

By the filtration in step (a) the antifoaming agent may be separated with the solid fraction while the molecule of interest may be obtained in the liquid fraction. Hence, fraction of the fermentation broth comprising the molecule of interest may be the liquid fraction, also referred to as permeate, and the second fraction comprising at least the antifoaming agent, also referred to as filter cake or retentate, may be the solid fraction.

The method and/or process according to the invention may further comprise a step (c) of subjecting the fraction of the fermentation broth comprising the molecule of interest obtained in step (b) to ultrafiltration, diafiltration and/or dia-ultrafiltration at a temperature below the cloud point of the antifoaming agent, preferably at a temperature of 2° C. to 18° C., more preferably at a temperature of 5° C. to 15° C.

A temperature below the cloud point of the antifoaming agent may be understood as a temperature that is at least 1° C. below the cloud point of the antifoaming agent, preferably at least 2° C., more preferably at least 5°, even more preferably at least 10° C. below the cloud point of the antifoaming agent. However, temperatures below the cloud point of the antifoaming agent should not be lower than 0° C. in order to avoid freezing damage of the cells in the fermentation broth or the molecule of interest. The temperatures below the cloud point of the antifoaming agent suitable for ultrafiltration, diafiltration and/or dia-ultrafiltration in step (c) may preferably refer to a temperature in the range of of 2° C. to 18° C., more preferably to a temperature of 5° C. to 15° C.

Step (c) may be performed subsequent to step (b). Step (c) may be followed by a step (d) of obtaining from step (c) a retentate comprising the molecule of interest and a permeate comprising at least parts of the antifoaming agent.

Step (c) may advantageously enhance the efficiency of the removal of the antifoaming agent. It is hypothesized that adjusting the temperature in optional step (c) to a temperature below the cloud point of the antifoaming agent may maintain the residual antifoaming agent in a solubilized form and lead to removal of residual antifoaming agent with the filtrate. Hence, step (c) may increase the efficiency of the separation of the antifoaming agent from the molecule of interest even further.

The removal of the antifoaming agent is preferably solely achieved by the filtration in step (b) and/or step (c). More preferably, the addition of a mineral clay or any other compound or molecule for complexing, binding or removing the antifoaming agent is not required in the method according to the invention. Advantageously, the entire method for separating a molecule of interest from an antifoaming agent in a fermentation broth comprising the molecule of interest and the antifoaming agent, e.g. steps (a) to (d), and potentially even further downstream processing steps, may be performed at temperatures of a maximum of 45° C. or even at a maximum of 40° C. or 30° C. such that heat-labile molecules of interest, in particular heat-labile enzymes, are protected and their function can be preserved. A further advantage is that the addition of a compound complexing or binding the antifoaming agent is not required. Therefore, the method according to the invention is simple and labor efficient.

In addition, the inventive method may comprise prior to step (a) the cultivation of the cells producing the molecule of interest. Said cultivation may be referred to as “fermentation process”.

The fermentation process may be of any known set-up, such as a batch process, a fed-batch process or a continuous fermentation process known to the skilled artisan.

Industrial fermentation processes are typically conducted by first providing a growth medium in a fermenter, inoculating the fermenter with an inoculum comprising cells capable of producing the molecule of interest and cultivating the cells under defined conditions such as pH, temperature, oxygen level etc., at a predefined time or until a predefined condition, e.g. titer, oxygen consumption, has been reached.

Thus, during the fermentation process, a cell producing the molecule of interest may be cultivated in a fermentation medium. Preferably, the cell is a microorganism, more preferably the cell is a recombinant microbial cell, even more preferably a recombinant bacterial cell.

The present invention may be useful for removing antifoaming agents from any fermentation broth in industrial scale, i.e., at least 1,000 liters, more preferably at least 5,000 liters, even more preferably at least 50,000 liters.

The method according to the present invention may comprise additional steps. The additional step may comprise the addition of compounds such as filtration aids/additives, activated charcoal, decolorants and the like.

These compounds may advantageously improve the separation of the molecule of interest and further enhance product quality.

Furthermore, the invention relates to a process for purifying a molecule of interest from a fermentation broth comprising the method for separating a molecule of interest from an antifoaming agent in a fermentation broth comprising a molecule of interest and an antifoaming agent.

The process of the invention may be applied to the fermentation broth or to a fraction of the fermentation broth. In particular, the process may comprise the method according to the invention including one or more additional step(s).

The process may comprise an additional step (e) of further purifying the molecule of interest, preferably by ion exchanging the fraction of the fermentation broth comprising the molecule of interest, more preferably by chromatography. In particular, the fraction of the fermentation broth comprising the molecule of interest obtained by separating the antifoaming agent from the molecule of interest in step (b) or as obtained in step (d) may be further treated by ion exchanging techniques.

These ion exchanging techniques may involve chromatography (e.g., ion exchange, affinity, hydrophobic, chromate-focusing, and size exclusion); electrophoretic procedures (e.g., preparative isoelectric focusing); differential solubility (e.g., ammonium sulfate precipitation); or extraction (see, e.g., Protein Purification, J.-C. Janson and Lars Ryden, editors, VCH Publishers, New York, 1989).

Still further, the process may comprise a step (f) of concentrating the molecule of interest, preferably by ultrafiltration, evaporation (such as thin film evaporation), precipitation, extraction, spray-drying, freeze-drying, or crystallization.

Still further, the process may comprise a step (g) of preparing a formulation containing the molecule of interest, preferably the protein of interest.

The formulations of the molecule of interest, in particular formulations of the protein of interest, can be either solid or liquid. Formulations can be obtained by using techniques known in the art. For instance, without being limited thereto, solid enzyme formulations can be obtained by extrusion or granulation. Suitable extrusion and granulation techniques are known in the art and are described for instance in WO 94/19444 A1 and WO 97/43482 A1. “Formulation of the molecule of interest” may refer to any non-complex formulation comprising a small number of ingredients, wherein the ingredients serve the purpose of stabilizing the molecule of interest comprised in the formulation and/or the stabilization of the formulation itself. The term “stability” relates to the retention of the activity of the molecule of interest as a function of time during storage or operation. The term “formulation stability” relates to the maintenance of physical appearance of the formulation during storage or operation as well as the avoidance of microbial contamination during storage or operation.

Preferably, the method of the present invention can be used for the manufacture of a purified or partially purified composition comprising the molecule of interest. More preferably, the method of the present invention provides the molecule of interest in purified or partially purified form.

Advantageously, it has been found by the inventors that antifoaming agents can be removed from a fermentation broth at relatively mild conditions, in particularly at mild temperatures, using the method described above which is suitable for the manufacture of heat-labile molecules of interest such as enzymes.

The explanations and interpretations of the terms made above apply mutatis mutandis to the embodiments described herein below.

The following embodiments are preferred embodiments of the method of the invention.

Embodiment 1. A method for separating a molecule of interest from an antifoaming agent in a fermentation broth comprising said molecule of interest and said antifoaming agent, the method comprising the following steps:
(a) filtering said fermentation broth at a temperature in the range of 1° C. to 15° C. above the cloud point of the antifoaming agent; and
(b) thereby obtaining a first fraction of the fermentation broth comprising the molecule of interest and a second fraction of the fermentation broth comprising the antifoaming agent, wherein the antifoaming agent is a polyalkylene glycol based antifoaming agent.

Embodiment 2. The method according to embodiment 1, wherein the antifoaming agent has a cloud point between 15° C. and 40° C., preferably a cloud point between 20° C. and 35° C.

Embodiment 3. The method according to any one of the preceding embodiments, wherein the temperature in the range of 1° C. to 15° C. above the cloud point of the antifoaming agent refers to a temperature in the range of 20° C. to 45° C.

Embodiment 4. The method according to any one of the preceding embodiments, wherein the antifoaming agent is selected from the list consisting of a polypropylene glycol (PPG), a polyethlyene glycol (PEG), polyalkylene glycol (PAG), an ethylene/propylene oxide block copolymer, a polyalcohol based on EO/PO block copolymer, an alkoxylated fatty acid ester, a polypropylene-polyethylene glycol copolymer (PPG-PEG), mixtures and derivatives thereof, preferably wherein the derivatives include ethers and co-polymers thereof.

Embodiment 5. The method according to any one of the preceding embodiments, wherein the antifoaming agent is selected from the list consisting of PPG, PEG, and PPG-PEG; preferably is a PPG having an average molar mass in the range of 500 to 5000 g/mol.

Embodiment 6. The method according to any one of the preceding embodiments, wherein the filtering in step (a) comprises microfiltration.

Embodiment 7. The method according to any one of the preceding embodiments, further comprising step (c) of subjecting the fraction of the fermentation broth comprising the molecule of interest obtained in step (b) to ultrafiltration, diafiltration and/or dia-ultrafiltration at a temperature below the cloud point of the antifoaming agent, preferably at a temperature of 2° C. to 18° C., more preferably at a temperature of 5° C. to 15° C.

Embodiment 8. The method according to any one of the preceding embodiments, wherein the molecule of interest is an enzyme.

Embodiment 9. The method according to any one of the preceding embodiments, wherein the molecule of interest is an enzyme selected from the group consisting of amylase, alpha-amylase, glucoamylase, pullulanase, protease, lipase, cutinase, acyl transferase, cellulase, endoglucanase, glucosidase, cellubiohydrolase, lactase, xylanase, xyloglucantransferase, xylosidase, mannanase, phytase, phosphatase, xylose isomerase, glucoase isomerase, acetolactate decarboxylase, pectinase, pectin methylesterase, polygalacturonidase, lyase, pectate lyase, arabinase, arabinofuranosidase, galactanase, laccase, peroxidase and an asparaginase, preferably wherein the enzyme is protease, amylase, lactase, or mannanase, more preferably, a protease, an amylase or a lactase, most preferably a subtilisin protease.

Embodiment 10. The method according to any one of the preceding embodiments, wherein the fermentation broth comprises recombinant or non-recombinant microbial cells, preferably bacterial cells, more preferably Bacillus cells.

Embodiment 11. The method according any one of the preceding embodiments, wherein the Bacillus cells are Bacillus licheniformis cells selected from the group consisting of Bacillus licheniformis as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.

Embodiment 12. The method according to any one of the preceding embodiments, wherein prior to step (a) the fermentation broth is obtained by cultivating microbial cells in a fermentation medium comprising the antifoaming agent.

Embodiment 13. The method according to any one of the preceding embodiments, wherein step (a) comprises cross flow filtration or dead-end filtration.

Embodiment 14. The method according to any one of embodiments 10 to 13, wherein the molecule of interest is secreted by the microbial cells into the fermentation broth.

Embodiment 15. A method for separating a molecule of interest from an antifoaming agent in a fermentation broth comprising said molecule of interest and said antifoaming agent, comprising the steps of
(a) obtaining a first fraction of the fermentation broth comprising the molecule of interest and a second fraction of the fermentation broth comprising the antifoaming agent by filtering said fermentation broth at a temperature in the range of 1° C. to 15° C. above the cloud point of the antifoaming agent; and
(b) thereby separating the molecule of interest from the antifoaming agent, wherein the antifoaming agent is a polyalkylene glycol based antifoaming agent.

Embodiment 16. The method of embodiment 15 having a feature of at least one of embodiments 2 to 14.

Embodiment 17. A process for purifying a molecule of interest from a fermentation broth comprising the method according to any one of the preceding embodiments.

Embodiment 18. The process according to embodiment 17, further comprising a step of further purifying the molecule of interest, preferably by ion exchanging the first fraction of the fermentation broth of step (b) comprising the molecule of interest, more preferably by chromatography.

Embodiment 19. The process according to any one of embodiments 15 to 18, further comprising a step of preparing a formulation containing the molecule of interest, preferably the molecule of interest being an enzyme.

The invention is further illustrated in the following examples which are not intended to be in any way limiting to the scope of the invention as claimed.

The present invention also relates to a composition comprising the molecule of interest obtainable by the method of the present invention.

All references cited throughout this specification are herewith incorporated by reference with respect to the specifically mentioned disclosure content and in their entireties.

### EXAMPLES

The invention will now be illustrated by working Examples. Theses working Examples must not construed, whatsoever, as limitations of the scope of the invention.

Unless otherwise stated the following experiments have been performed by applying standard equipment, methods, chemicals, and biochemicals as used in genetic engineering and fermentative production of chemical compounds by cultivation of microorganisms. See also Sambrook et al. (Molecular Cloning: A Laboratory Manual. 2nd edition, Cold Spring Harbor Laboratory, Cold Spring Harbor Laboratory Press, Cold Spring Harbor, N Y, 1989) and Chmiel et al. (Bioprocesstechnik 1. Einführung in die Bioverfahrenstechnik, Gustav Fischer Verlag, Stuttgart, 1991).

The fermentation broths for the examples below (Examples 1-3) were obtained by culturing Bacillus licheniformis cells comprising a gene coding for a subtilisin protease. Bacillus licheniformis cells were cultivated in a fermentation process using a chemically defined fermentation medium providing the components listed in Table 1 and Table 2.

TABLE 1
Macroelements provided during the course of the
fermentation process

| | | Concentration |
|---|---|---|
| | | [g/L initial |
| Compound | Formula | volume] |
| Citric acid | C₆H₈O₇ | 3.0 |
| Calcium sulfate | CaSO₄ | 0.7 |
| Monopotassium phosphate | KH₂PO₄ | 25 |
| Magnesium sulfate | MgSO₄*7H₂O | 4.8 |
| Sodium hydroxide | NaOH | 4.0 |
| Ammonia | NH₃ | 1.3 |

TABLE 2
Trace elements provided during the
course of the fermentation process

| Trace | | Concentration |
|---|---|---|
| element | Symbol | [mM] |
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

A solution containing 50% glucose was used as feed solution. pH was adjusted during fermentation using ammonia. At the desired product titer the fermentation was terminated, and the product amylase was present in both soluble and crystalline form as confirmed by visual inspection using a microscope. At the end of fermentation, the phosphate concentration was about 3 g/L.

### Example 1: Experimental Determination of Cloud Point of Pluriol® P2000 (a PPG Having an Average Molar Mass of Approx. 2000 g/Mol)

600 mL of deionized water was cooled to about 5° C. in an ice bath and a corresponding amount of Pluriol® P2000 was added to adjust its concentration to 0.1, 1.0 or 2.5 g/L. The solution was emulsified using a Polytron homogenizer at 7500 rpm for 10 min. After a few minutes the resulting air bubbles disappeared and the clear solution was slowly warmed under constant gentle stirring. Samples (15 mL) were taken at certain temperature points and the turbidity was measured using a portable turbidimeter 2100Q from Hach turbidity. A strong increase in turbidity as a function of temperature indicated the observed cloud point temperature (Tcp).

TABLE 3

| Concentration | |
|---|---|
| of Pluriol ® | Observed |
| P2000 | Top |
| [g/L] | [° C.] |
| 0.1 | 27-28 |
| 1.0 | 22-23 |
| 2.5 | 20-21 |

### Example 2: Microfiltration of a Fermentation Broth of B. licheniformis Comprising Antifoaming Agent at Various Temperatures

The antifoaming agent present in the fermentation broth was Pluriol® P2000 (a polypropylene glycol commercially available from BASF) with a cloud point in the range of 20° C. to 27° C. as determined in example 1 above. Dead-end microfiltration was performed using a 0.2 μm filter and device from Pall Corporation Pall Filter Supor EKV membrane—KA3EKVP6G. Various fractions of a fermentation broth of B. licheniformis, biomass-free, were subjected to microfiltration. Although post-processing steps for removing the biomass were already conducted, the amount of antifoaming agents was still significant (see Table 4).

The amount of antifoaming agent in the respective fractions was determined at room temperature. The amount of the antifoaming agent was determined by H PLC-MS analysis using a Single Quad MS LCMS device. The device was a Shimadzu Nexera; Single-Quad MS LCMS2020; the HPLC column was a Kinetex C18; 2.1×100 mm; 1.7 μm; the solvent was A: water; 0.1% HCOOH and B: acetonitrile/isopropanol (50/50); 0.1% HCOOH; the quantification was performed using the triple charged species of the ten highest signals.

TABLE 4

| | | Anti- |
|---|---|---|
| | | foaming |
| | | agent |
| | Fraction | [mg/ml] |
| Batch | Starting material: fermentation broth, | 1.46 |
| 1-1 | biomass-free | |
| | Filtrate obtained by filtering at 6 to 15° C. | 1.12 |
| Batch | Starting material: fermentation broth, | 1.64 |
| 1-2 | biomass-free | |
| | Filtrate obtained by filtering at 35° C. | 0.44 |
| Batch | Starting material: fermentation broth, | 0.44 |
| 2-1 | biomass-free | |
| | Filtrate obtained by filtering at 6 to 15° C. | 0.46 |
| Batch | Starting material: fermentation broth, | 0.42 |
| 2-2 | biomass-free | |
| | Filtrate obtained by filtering at 35° C. | 0.18 |

It can be seen that filtration at a temperature above the cloud point of Pluriol® P2000, e.g. at 35° C., leads to efficient depletion of residual antifoaming agent from the fermentation broth, while filtration at lower temperatures, e.g. 6° C. to 15° C., has only marginal effects on the amount of residual antifoaming agent in the fermentation broth.

### Example 3: Depletion of Antifoaming Agent and Biomass from a Fermentation Broth of B. licheniformis Comprising Protease

Pluriol® P2000 was used as the antifoaming agent. The amount of antifoaming agent was determined as in example 2. The fermentation broth was centrifuged in a laboratory centrifuge at 10000 g for 10 min to obtain the supernatant.

This example demonstrates that the antifoaming agent can be successfully removed or at least largely depleted from the fermentation broth during biomass separation. Hence, biomass and antifoaming agent may be removed together.

TABLE 5

| | | | | | Total anti- |
|---|---|---|---|---|---|
| | | Anti- | Batch vol. | Explanation | foaming |
| | Temp. of | foaming | after prec. | concerning | agent in |
| | prec. DSP st. | agent | DSP st. | vol. of | sample |
| Sample | [° C.] | [mg/ml] | [kg] | sample | [g] |
| Supernatant of | 30 | 3.1 | 12 | prior to MF | 37.2 |
| fermentation | (fermentation) | | | | |
| broth | | | | | |
| Pool permeate | 30 (MF) | 0.057 | 46.74 | vol. of | 2.66 |
| after MF | | | | permeate | |
| | | | | after MF | |
| Pool retentate | 10 (UF) | 0.142 | 0.86 | after | 0.12 |
| after UF (prior to | | | | concentrating | |
| diafiltration, after | | | | | |
| concentrating) | | | | | |
| Pool retentate | 10 (UF) | 0.066 | 0.57 | washed with | 0.04 |
| after dia-UF | | | | 1.49 kg, | |
| | | | | concentrated | |

prec. DSP st.-preceding downstream processing step
vol.-volume

Performing the microfiltration (MF) at a temperature above the CP (e.g. at 30° C.) results in efficient removal of the major part of the antifoaming agent. The permeate of the microfiltration comprises the molecule of interest (enzyme) and significantly less amount of antifoaming agent compared to the fermentation broth. Subsequent ultrafiltration (UF) and dia-ultrafiltration at a temperature below the CP of the antifoaming agent results in further depletion of the residual antifoaming agent together with the permeate, while the enzyme retains in the retentate and may be subjected to further purification steps such as chromatography or a second ultrafiltration. The amounts of antifoaming agents in the permeate of the microfiltration and in the ultrafiltration-retentate are significantly reduced. As a further advantage, fouling of the ultrafiltration membrane and during chromatography in subsequent purification steps can be avoided.

1. A method for separating a molecule of interest from an antifoaming agent in a fermentation broth comprising the molecule of interest and the antifoaming agent, the method comprising:
(a) filtering the fermentation broth at a temperature in the range of 1° C. to 15° C. above the cloud point of the antifoaming agent; and
(b) thereby obtaining a first fraction of the fermentation broth comprising the molecule of interest and a second fraction of the fermentation broth comprising the antifoaming agent,
wherein the antifoaming agent is a polyalkylene glycol based antifoaming agent.

2. The method according to claim 1, wherein the antifoaming agent has a cloud point between 15° C. and 40°.

3. The method according to claim 1, wherein the temperature in step (a) in the range of 1° C. to 15° C. above the cloud point of the antifoaming agent, is a temperature in the range of 20° C. to 45° C.

4. The method according to claim 1, wherein the antifoaming agent is selected from the group consisting of a polypropylene glycol (PPG), a polyethlyene glycol (PEG), polyalkylene glycol (PAG), an ethylene/propylene oxide block copolymer, a polyalcohol based on EO/PO block copolymer, an alkoxylated fatty acid ester, a polypropylene-polyethylene glycol copolymer (PPG-PEG), mixtures thereof, and derivatives thereof.

5. The method according to claim 1, wherein the antifoaming agent is selected from the group consisting of PPG, PEG, and PPG-PEG.

6. The method according to claim 1, wherein the filtering in step (a) comprises microfiltration.

7. The method according to claim 1, further comprising a step (c) of subjecting the first fraction of the fermentation broth comprising the molecule of interest obtained in step (b) to ultrafiltration, diafiltration, and/or dia-ultrafiltration at a temperature below the cloud point of the antifoaming agent.

8. The method according to claim 1, wherein the molecule of interest is an enzyme.

9. The method according to claim 1, wherein the molecule of interest is an enzyme selected from the group consisting of amylase, alpha-amylase, glucoamylase, pullulanase, protease, lipase, cutinase, acyl transferase, cellulase, endoglucanase, glucosidase, cellubiohydrolase, lactase, xylanase, xyloglucantransferase, xylosidase, mannanase, phytase, phosphatase, xylose isomerase, glucoase isomerase, acetolactate decarboxylase, pectinase, pectin methylesterase, polygalacturonidase, lyase, pectate lyase, arabinase, arabinofuranosidase, galactanase, laccase, peroxidase, and an asparaginase.

10. The method according to claim 1, wherein the fermentation broth comprises recombinant or non-recombinant microbial cells capable of producing the molecule of interest.

11. The method according to claim 1, wherein prior to step (a) the fermentation broth is obtained by cultivating microbial cells in a fermentation medium comprising the antifoaming agent.

12. The method according to claim 1, wherein step (a) comprises cross flow filtration or dead-end filtration.

13. The method according to claim 10, wherein the molecule of interest is secreted by the microbial cells into the fermentation broth.

14. A process for purifying a molecule of interest from a fermentation broth comprising the method steps according to claim 1.

15. The process according to claim 14, further comprising a step of further purifying the molecule of interest, by ion exchanging the first fraction of the fermentation broth of step (b) comprising the molecule of interest.

16. The process according to claim 14, further comprising a step of preparing a formulation containing the molecule of interest.

17. The method according to claim 5 wherein the antifoaming agent is a PPG having an average molecular mass in the range of 500 to 5000 g/mol.

18. The method according to claim 7 wherein the temperature below the cloud point of the antifoaming agent is 2° C. to 18° C.

19. The method according to claim 10 wherein the microbial cells are bacteria cells.
